(19) United States
(12) Patent Application Publication
(10) Pub. No.: US 2026/0348919 A1
(43) Pub. Date: Nov. 5, 2026

(54) QUANTUM DOTS SENSING CONTRAST AGENT FOR MONITORING CO2 AND ITS MANUFACTURING THEREOF

(71) Applicants: Feipeng LIU, SPRING, TX (US); Yuning LAI, SPRING, TX (US)

(72) Inventors: Feipeng LIU, SPRING, TX (US); Yuning LAI, SPRING, TX (US)

(21) Appl. No.: 19/282,163

(22) Filed: Jul. 28, 2025

Related U.S. Application Data
(60) Provisional application No. 63/686,443, filed on Aug. 23, 2024.

(51) Int. Cl. G01N 29/02; B82Y 15/00; B82Y 20/00; G01N 29/14; G01N 29/24; G01N 33/00

(52) U.S. Cl. CPC G01N 29/022; B82Y 15/00; B82Y 20/00; G01N 29/14; G01N 29/2418; G01N 29/2443; G01N 33/004; G01N 2291/02809

(57) ABSTRACT

The current electromagnetic (EM) methods are well suited for monitoring CO₂ in carbon capture and storage (CCS) in sequestration of hydraulic fracking operation; however, it is less efficient, discontinuous, disclosed here, a quantum dots (QDs) sensing Contrast (QDSCA) emitter for continuously monitoring CO₂ emission 24/7 onsite in carbon capture, storage (CCS) comprised of: 1) Ag₂S quantum dots, 2) microchannels, 3) an integrated digital transducer (IDT) board, 4) hydrogel polymers, fabricated on a micro-crystalline piezoelectric quartz substrate, functionalized by a photo-illumination laser, excited at a wavelength of between 800 to 900 (nm) to convert photo energy into a short-wave infrared (SWIR) from 1000 to 1700 (nm) in imaging from 1000 to 1700 (nm) plus a strong absorption band of CO₂ at 2000 to 2500 (nm) detectable by an ultrasonic scanner in a multi-sensing mode of radio frequency (RF), surface acoustic wave (SAW), SWIR that can be detected by ultra frequency spectrums.

### CROSS REFERENCE TO RELATED APPLICATIONS

U.S. provisional patent application of 63/732,443 filed on Aug. 15, 2024, of 63/684,443 on Aug. 19, 2024, both applications are continuation in part of U.S. applications of claims priority US patent application of Ser. No. 17/452,565 (filed on Oct. 2, 2023).

### STATEMENT

None of these research projects has been funded by U.S. Government Grants.

### FIELD OF INVENTION

This invention is related to developing a quantum dot sensing contrast agent for monitoring the CO₂ emission in carbon capture, storage, and in sequestration (CCS) as a multi-mode signal emitter. The target markets include the type VI CO₂ injection wells and type II waste wells from oil and gas explorations, and CO₂ fracking wells for oil and gas in the subterrain operation. The purpose of the developed monitoring tools is to tackle climate change after CO₂ wells dried for monitoring the emission of CO₂ continuously. In addition, it is estimated that the CO₂-EOR fracking can increase oil production by as much as 50 percent and get claimed climate credits per metric ton of CO₂ sequestrated. Concerning the safety of CO₂ onsite emissions to unconfined areas for underground aquifer system requires the operators to monitor, verify, and account for the CO₂ emission constantly. The developed monitoring system will allow CO₂ plume to be continuously monitored 24/7 on site. Technically, it is also suitable for applying the developed products in re-fracking and newly drilled oil and gas wells.

### BACKGROUND OF INVENTION

The global carbon capture and sequestration (CCS) market size was valued at USD 2.69 billion in 2023. It is projected to grow from USD 3.54 Billion in 2024 to USD 14.5 Billion by 2032 at a CAGR rate of 19.29% during the forecast period. The capture of carbon dioxide is classified by methods such as enhanced oil recovery (EOR) and CO₂ emission, directly injected into CCS wells, converting fertilizer and biofuels. Large collaboration among different industry participants in large scale projects for cost share projects with long-term sustainable growth has been intensified with huge investments around the world. In March 2023, the net-zero industry was introduced by the European Union to target an annual objective of 50 million metric tons of CO₂ per year by 2030.

In U.S., support on net-zero of carbon emission has been driven by the incentive of DOE and EPA efforts to tackle climate change and reach a net-zero emission economics by 2050. The CO₂-EOR fracking can increase a well's oil production by as much as 50 percent and get claimed climate credits from USD 60 to 130 per metric ton of CO₂ sequestrated, however, knowing how much carbon dioxide sequestrated in EOR is far from satisfactory with a potential for abuse which adds another layer of urgency based upon the energy news.

In one embodiment of CO₂ sequestration, the most import and difficult to eliminate is CO₂ from coal mining plants. In this case, the CO₂ generated from coal plants will be pipelined to a type VI well miles away from the coal power plants, then, injected with a downhole compressed pressure. The operational cost of sequestrating the CO₂ is very high. Concern on the safety of CO₂ onsite emissions to unconfined areas for underground aquifer system requires the operator to monitor, verify, and account for the CO₂ emission, however, there is no available method to do so. The current electromagnetic (EM) methods are suited for monitoring CO₂ in sequestration, however, factors of controlling the imaging signal to noise (SNR) are not clearly defined, not continuous. New monitoring tools with continuous modes for CO₂ plume are needed.

In one embodiment of product applications, technically, although it is targeted for the type VI well and CO₂-EOR well of the CO₂ injection, it is also suitable for applying the products in re-fracking and newly drilled wells.

In re-fracking situation, the re-fracking, also known as re-fracturing, is the process of stimulating an existing oil or gas well that has already undergone hydraulic fracturing (fracking) to increase production. It involves re-injecting fluids (often with diverting agents or other modifications) into the wellbore to create new or reopen existing fractures in the surrounding rock, thus increasing the flow of hydrocarbons to the well.

Hydraulic fracturing involves injecting high-pressure water, sand, and chemicals through a wellbore to fracture surrounding rock. This process facilitates the flow of oil and gas. Over time, the initial fractures may close or become less effective, leading to a decline in production from the well. Re-fracking aims to address this decline by stimulating the well again, involving a) re-injecting fluid to reopen or widen existing fractures, b) injecting fluid at higher pressures or with different additives to create new fractures in the rock, c) implementing materials that can selectively block existing fractures, d) forcing the fracturing fluid to stimulate new areas, e) creating new or enhanced pathways for hydrocarbons to flow. The re-fracking can revitalize older wells that have seen a decline in production, extending their productive lifespan, and potentially increasing the overall recovery of resources.

In the case of CO₂ fracking operation in the hydraulic fracturing scenario, CO₂ is injected into oil formations, where it mixes with and helps mobilize trapped oil. This process can significantly increase the recovery of oil, sometimes even bringing up more oil than the initial drilling phase called CO₂-enhanced oil recovery (EOR).

The benefits of EOR-CO₂ fracking include: a) enhanced Oil Production since CO₂ acts as a lubricant and thins the oil, allowing it to flow more easily; b) carbon sequestration: a significant portion of the injected CO₂ remains underground, which is viewed as a beneficial method for carbon capture and storage (CCS); c) in Texas, fluid injection into oil and gas reservoirs, including CO₂ injection, is governed by the Railroad Commission of Texas under Statewide Rule 46. d) Direct air capture (DAC) and carbon sequestration projects are also involved in CO₂-EOR, like other oil and gas activities, presents potential environmental risks including groundwater contamination and induced seismicity. e) concerns exist regarding the effectiveness of regulations in mitigating these risks, particularly regarding potential leaks from wells.

In summary, the use of CO₂-EOR, potentially combined with other technologies like chemical injections or nanoparticle additives, is seen as a promising path for increasing oil recovery rates and reducing carbon emissions.

In terms of financial feasibility, the development products can be directly applied to current CO₂-EOR fracking well applications. Once the application of developed technologies is approved, the CO₂-EOR fracking can support the long-term net-zero goal for type VI wells that directly pump the CO₂ from coal power plants. In terms of applying the products to type VI wells, it may take a long time to work with other big companies, however, the financial incentive from the collaboration with local communities and government entities is also great.

Regarding Class VI wells, carbon dioxide (CO₂) injected into deep rock formations has been extensively tested by scale-up tests. This long-term underground storage is called geologic sequestration (GS). Geologic sequestration as part of carbon capture and storage (CCS) is a technology that can be used to reduce CO₂ emissions to the atmosphere. Possible sources of CO₂ for GS include CO₂ captured from point source emissions such as from an industrial facility (e.g., steel and cement production) or energy production (e.g., ethanol, hydrogen production, or power plants), as well as CO₂ captured directly from the atmosphere.

The safety requirements for these wells focus on protecting underground sources of drinking water (USDWs) and public health. Some key safety considerations include a) ensuring that the geology can securely contain CO₂ without risks like fractures or induced seismicity; b) accounting for its buoyancy, mobility, and corrosivity when mixed with water; c) managing large amounts of CO₂ safely; d) continuous oversight to detect any potential leaks or pressure changes.

Currently, class VI uses advanced monitoring techniques to detect and prevent failures. Some key methods include a) continuous tracking of injection pressure to identify anomalies; b) groundwater sampling; c) regular testing to ensure that CO₂ hasn't leaked into underground sources of drinking water; d) seismic monitoring to detect small earthquakes that could indicate geological instability; e) CO₂ plume tracking with computing models and sensors to follow the movement of injected CO₂; f) checking casing and cement conditions to prevent leaks. EPA has published special guidance for life cycle of class III project: 1) pre-permitting; 2) pre-construction; 3) pre-operation; 4) injection; 5) post-injection.

Failure in Class VI wells can have serious environmental and safety implications. Some key concerns include: 1) if CO₂ leaks from the well, it can mix with underground water sources, potentially making them unsafe for consumption; 2) poorly managed injection can lead to induced earthquakes, which may damage infrastructure or disrupt local communities; 3) health risks of CO₂ leaking that can pose asphyxiation hazards in confined spaces, affecting workers or nearby residents; 4) regulatory consequences of operators that may face fines, shutdowns, or legal action if they fail to comply with safety regulations.

There are various methods available for monitoring CO₂ emissions. The current electromagnetic (EM) methods are well suited for monitoring CO₂ in sequestration, however, factors of controlling the imaging signal to noise (SNR) are not clearly defined. Verification on what happens under the subterraneous formation is impossible due to the formation variation in porous media, density, and chemical composition. In summary, the EM and seismic service are expensive but less effective, in contrast, monitoring the CO₂ emission with bio-based materials as a labelling agent with short-wave infrared-red (SWRI) provides an easy and practical approach for imaging 24/7.

Quantum dots exhibit size-tunable emission and can be chemically functionalized to bind CO₂ molecules. Photoluminescence (PL) shifts caused by analyte interaction allow for optical signal transduction with molecular Sensity. Integration into micro-scale systems makes QDs ideal for embedded environmental monitoring platforms.

Surface acoustic waves (SAW), generated via interdigital transducers on piezoelectric substrates (e.g., LiNbO₃) for dynamic modulation produce strain and electric fields that influence QD emission. These interactions enhance sensitivity and enable modulation of the optical response. SAW-driven modulation of QDs has been validated with 2D materials and semiconductor nanowires.

Radio frequence (RF) control for tunning and signal processing systems serve as the control and feedback mechanism, driving SAW generation and dynamically tunning resonances.

The demand for monitoring CO₂ accurately is growing across industrial and environmental sectors. Traditional gas sensors often suffer from bulkiness or limited selectivity. Currently, quantum dot materials offer a new paradigm in sensing technologies due to their optical tunability, and high chemical sensitivity are needed.

### BRIEF SUMMARY OF INVENTION

A quantum dots (QD) sensing contrast (QDSCA) agent is disclosed here, comprised of: 1) a microcrystalline silica sand, 2) partially coated with a metallic layer; 3) tunable microchannels filled with Ag₂S QDs arrays; 4) an integrated logic conductive wired network to emit radio frequency; 5) multi-layered hydrogel polymers, fabricated into a smart device by receiving photonic energy from an illuminated laser excited at 800 to 900 (nm) with up irradiation bands of QDs around 1100 and 1650 (nm) to fingerprint the QDSCA sample identification in situ with a shortwave Infrared (SWIR) imaging camera and a strong CO₂ absorption band at around 2000-2500 (nm) of CO₂ volumetric expansion in the micro-channels of determining the CO₂ concentration of a noninvasive ultrasonic detector using the encapsulated CO₂ as a contrast agent and the emitter of the radio frequency (RF) from the integrated logic (IL) as an antenna in the CO₂ capture and storage sequestration application.

FIG. 1 illustrates a quantum dot sensing contrast agent (QDSCA) product used to measure CO₂ concentration in situ during carbon capture and storage (CCS). It consists of (a) a microcrystalline silica; (b) a metallic layer; (c) tunable channels filled with a metal oxide of QDs (1-c1) and microlenses (1-d1) made of quantum dot's arrays, (d) conductive wires inserted with carbon QDS (1-b2) encapsulated in a hydrogel polymer coating as a signal emitter with unique identities assigned to each particle.

In the design, QDSCA will first receive photonic energy from an illuminated laser excited at 800 to 900 (nm), then, subsequentially store the energy at an up conversion of irradiation bands between around 1100 and 2500 (nm) for organic carbon imaging and generate a strong CO₂ absorption band at 2000-2050 (nm) stored in six designated micro-channels (1-c1). Once the electron charge energy at the irradiation bands is above the band gap energy threshold value, the QD's particles will discharge themselves at a special illumination decay rate. The image captured by the SWIR camera frame in a fixed interval will be used to fingerprint a barcode for identifying the sample's position, then, the In-situ imaging will be used to calculate the moving rate of digitalized pixels extracted from a database of pixels pi (x, y, z) (m=1, 2 . . . n). The plume movement in situ can be calculated by equation 1:

$${\sum }_{m = 1}^{n} {( \frac{Δ p}{Δ t} )}_{m} = \frac{Δ p}{Δ x} i + \frac{Δ p}{Δ y} j + \frac{Δ p}{Δ z} k … … ( m = 1 , 2 , … … n ) \qquad ( 1 )$$

where i, j, k, the direction of 3-d vectors, p(x,y,z)ₘ the central point of the position of each QDSCA imaging, m the number of identified pixels.

As SWIR imaging exhibits reduced interference from water, oil, and other impurities, along with enhanced penetration and defined resolution, it offers superior localized signal-to-noise ratio (SNR) compared to EM imaging. By modulating the QDSCA as an emitter (m), pressure varies with a well-defined frequency created wave. CO₂ concentration is calculated using signal-processing algorithms (m=1, 2, 3 . . . n). The CO₂ plume velocity from surface acoustic waves (Vco2) will be applied to Wyllie's velocity-volume approximation for sedimentary rocks, as shown in equation 2:

$$\frac{1}{{V}_{p}^{m}} = \frac{1 - ∅}{{V}_{p max}^{m}} + \frac{{S}_{ft}}{{V}_{CO 2}^{m}} + \frac{( 1 - {S}_{ft} )}{{V}_{p w}^{m}} \qquad ( 2 )$$

where

$${V}_{p}^{m} , {V}_{p max}^{m} , {V}_{p w}^{m}$$

are the p-wave velocities of the saturated rocks, the rock matrix, the pore fluid (CO₂ plume) represented by QDSCA emitter, and salt water fluid, respectively, S_{ft}, a fitting constant, Φ is the pore space volume.

An integration of

$${V}_{p}^{m}$$

into CSEM models will create an onsite 3D CO₂ emission model in 24/7 mode. The drift of CO₂ plume profile from geological surveys' data can also be determined.

A typical design of a QDSCA product based upon the above strategy is shown in FIG. 2, comprised of a surface acoustic wave (SAW) chip with an interdigital transducer (IDT) deposited on a piezoelectric quartz surface to generate SAWs for enabling multifunctional sensing of strain, temperature, and vibration. QDs semiconductors with bio-based carbon dots will be randomly deposited on the IDT conductive wires. Materials such as silver sulfide (Ag₂S), CuO, CuS, and other metal quantum dots such as TiO₂, ZnO, TiO₂, CrO, TaO, GaAsAl_{0.3}Ga_{0.7}, etc., will be used to produce the QDs. Since quartz can convert mechanical energy to electrical energy, QDSCA can vibrate itself.

Another typical design of A QDSCA product based upon the above strategy is shown in FIG. 3, comprised of a micro-ring, which possess circular boundaries to support whispering gallery mode (WGM) resonators that can be used as a signal emitter, consisting of a piezoelectric micro-ring made of quartz, or other piezoelectric materials, a hydrogel coating encapsulated with quantum dots (QDs) such as soy protein isolate (SPI) organic or Ag₂S nanoparticles.

Another typical example is also included in FIG. 4, The product served as an emitter, comprised of a micro quartz disk having a diameter of D, on the top of quartz disk, micro-printing with copper conductor wired patterns, having different antennal length based upon the connected pattern of a1, a2, a3, a4, b, and c. The size of the disk diameter is less than 1000 (micron) that can be printed with a conductive pattern utilizing a conductive copper paste. The paste was dried and dried paste was heated under 700 to 850 °C before being coated with a layer of hydrogel coating in which dual nano dots domain (SPI or Ag₂S) will be encapsulated within the coatings.

Another typical example is also included in FIG. 5. The product served as a spectrum emitter, comprised of a layer or multiple layered quantum dots, sintered on the bottom layer of piezoelectric layers such as microcrystalline quartz, interfaced with a layer of integrated digital transducer (IDT). The quantum dots create a porous membrane layer with a special permeable rate. The migration rate of CO₂ gas and its concentration can be monitored via the integrated digital transducer (IDT). The value of CO₂ concentration can be monitored online via the circuitry of radio frequency of cumulated circular boards.

The above QDs semiconductors exhibit size-tunable emission and can be chemically functionalized to bind CO₂ molecules. Photoluminescence (PL) shifts caused by analyte interaction allow for optical signal transduction with molecular sensitivity. Integration into micro-scale systems makes QDs ideal for embedded environmental monitoring platforms.

In one embodiment of the inventions, the quantum dots made of semiconductor nanocrystals are semiconductor particles in a few nanometers in size with optical and electronic properties that differ from those of larger particles via quantum mechanical effects. When a quantum dot is illuminated by UV light, an electron in the quantum dot can be excited to a state of higher energy. In the case of a semiconducting quantum dot, this process corresponds to the transition of an electron from the valence band to the conduction band. The excited electron can drop back into the valence band releasing its energy as light. This light emission (photoluminescence) is illustrated in the figure on the right. The color of that light depends on the energy difference between the discrete energy levels of the quantum dot in the conduction band and the valence band at a wavelength between 700 nm to 2500 nm.

Alternatively, quantum dot can be defined as a structure on a semiconductor which is capable of confining electrons in three dimensions, enabling the ability to define discrete energy levels. The quantum dots are tiny crystals that can behave as individual atoms, and their properties can be manipulated.

Nanoscale materials with semiconductor properties tightly confine either electrons or electron holes. The confinement is similar to a three-dimensional particle in a box model. The quantum dot absorption and emission features correspond to transitions between discrete quantum mechanically allowed energy levels in the box that are reminiscent of atomic spectra.

Quantum dots have properties intermediate between bulk semiconductors and discrete atoms or molecules. Their optoelectronic properties change as a function of both size and shape. Larger QDs of 5-6 nm diameter emit longer wavelengths, with colors such as orange, or red. Smaller QDs (2-3 nm) emit shorter wavelengths, yielding colors like blue and green. However, the specific colors vary depending on the exact composition of the QD.

Potential applications of quantum dots include single-electron transistors, solar cells, LEDs, lasers, single-photon sources, second-harmonic generation, quantum computing, cell biology research, microscopy, and medical imaging. Their small size allows for some QDs to be suspended in solution, which may lead to their use in inkjet printing, and spin coating. They have been used in Langmuir-Blodgett thin films. These processing techniques result in less expensive and less time-consuming methods of semiconductor fabrication.

In another embodiment of the invention, the substrate in a likely shape of disks or particles is comprised of quartz, bauxites, sand, particles, or synthesized piezoelectric materials of barium titanate (BaTiO₃) nanoparticles (BTNPs), titanium oxides (TIO₂). The size of these piezoelectric materials has been tailored prepared within targeted in a diameter of less than 3000 (micron), or one thousand (micron). The optimal quartz material possesses a diameter of approximately 600 microns or less.

In another embodiment of the invention, the preferred material compositions comprise at least metal halide and the combination of one or more metals with at least one metal halide. Preferred metals include copper, zinc, silver, and their alloys—particularly copper iodide, copper(I) chloride, silver iodide, silver bromide, and silver chloride. Examples of these alloys are those of silver copper, copper+tin(bronze) and copper zinc (Brass is an alloy of copper and zinc with typical copper concentration in the range of 40 to 90% by weight, and may have additional elements, e.g., as in phosphor bronze). Metallic oxide compounds are also preferred materials such as magnesium oxide, zinc oxide, aluminum oxide, etc.

In another embodiment of the invention, copper salts may have a range of water solubility characteristics. However, it is preferred that the copper salts of the present invention have low water solubility so that they may have slow and predictable copper cation release characteristics. In some formulations, it may be desirable to also add Cu (II) or more soluble salts so that some fractions of Cu ions are quickly available. Cu(I) cations have shown the most efficacy. At room temperature, copper salts with solubilities below 100 mg/liter are preferred, especially those under 15 mg/liter.

In many applications lower water solubility is important. In some cases, metal salts with high water solubility are added to release a high concentration of ions over a short period of time.

In another embodiment, the short-wave infrared spectrum (SWIR) camera on imaging of CO₂ plumes from strong point sources has been used to retrieve the CO₂ absorption structures around 2000 (nm) wavelength, which is analog to previously reported satellite techniques that work. A commercial SWIR camera can measure absorption intensity at 2000 nm, enabling CO₂ concentration determination via a pre-calibration chart with less than 25% uncertainty.

### BRIEF DESCRIPTION OF THE DRAWINGS

FIG. 1. Schematic of proposed quantum dots sensing contrast agent (QDSCA) for monitoring CO₂ emission. The product like a drum disk is an emitter, stimulated by as laser/LEDs light. It can transmit multi-sensing signals to microphone, SWIR camera, and computers via an acquisition board. 1-a0: silica sand or proppants, bauxite of macro/micro particles; 1-b1: controllable macro/micro metallic layer; 1-b2: Ag₂S quantum dots (A semiconductor material); 1-b3: carbon quantum dots (made of AquluMax™); 1-c1—Microlens and drilled micro-channels for absorbing CO₂; 1-d1—multilayered hydrogel that provides the protection and an In situ or vivo formation of quantum dots.

FIG. 2. Schematic of the design of QDSCA emitter: 1) layout of SAW device design: the chip width, the IDT aperture, the interdigital transducer (IDT) length, and the chip length for SAW propagation and attenuation in fluid as W, a, l, and L, respectively; 2) quantum dots of semiconductors hybrid with carbon dots on the top of chip channels; 3) wired antenna that transfers the RF signals; 4) micro-channel for generating bubbles in the QDSCA emitter to enhance the contrast in PAW; 5) A curable hydrogel coating that can seal the QDSCA; 6) substrate of surfaced 20/40 quarts particles having a thickness of 300 (nm) and diameter of 600 (nm).

FIG. 3 Schematic of quantum dots sensing contrast (QDSCA) agent for monitoring CO₂. It can transmit multi-sensing signals to microphone, SWIR camera, and computers via an acquisition board. The schematic of a micro-ring quart disk has a diameter of D coated with a thin layer of hydrogel coating (thickness t). The diameter of inner holes is d. The thickness of the micro-ring is H. The diameter of quantum dots is 2r widely spread on both sides of the disks.

FIG. 4A schematic of piezoelectric substrate with flatten surface printed with integrated digital transducer (IDT) pattern, quantum dots (QD), then, sintered on the top of IDT and the surface of substrates.

FIG. 5. A schematic of a disk-shaped piezoelectric substrate with a 3d-printing circulatory maze pattern (a1, a2, a3, b, c) as RF antenna printed on the surface as intermediate IDT spread with quantum dots particles.

FIG. 6. QDSC emitter schematics for different scenarios: a) beneath the surface at downhole distances of 5,000-9,000 ft, sealed in cap rock; b) beneath sub-sea-floor beds, c) schematic of CO₂ plume in sealed injected well (case I, II, III), d) schematic of QDSCA for monitoring gas and others with multi-sensing mode (photo-diol laser, RF, PAI, and SWIR imaging)

FIG. 7. Transmission spectra of a hydrogel coated micro-ring quartz at input powers of 0.70 m/W (a) and 3.1 mW (b). The spectrum is corresponded of SWIR spectrum above 1000 (nm).

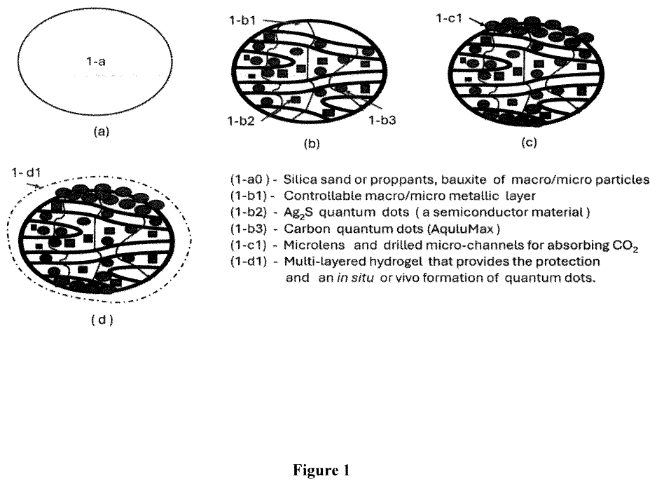

### DETAILED DESCRIPTION OF EMBODIMENTS OF THE INVENTION

Carbon capture and storage (CCS) is essential for net-zero emissions by cutting industrial CO2, enabling clean hydrogen, and converting atmospheric carbon into useful chemicals. Procedures for confinement of CO₂ leaking include establishing a baseline on sequestration permits and reconfirming the surrounding area unaffected by injected CO₂. The current electromagnetic (EM) methods are well suited for monitoring CO₂ in sequestration, however, factors of controlling the imaging signal to noise (SNR) are not clearly defined. Verification on what happens under the subterraneous formation is impossible due to the formation variation in porous media, density, and chemical composition. In summary, the EM and seismic service are expensive but less effective, in contrast, monitoring biobased materials such as CO₂ plume with short-wave infrared-red (SWRI) provides an easy and practical approach for imaging (Thimsen, E. T., B. Sadtler, and M. Berezin 2017).

Like other optical wave functions, short-wave infrared-red (SWIR) spectrum has been characterized by the following Brus's equation (3):

$${E}_{g}^{+} ( eV ) = {E}_{g}^{b u l k} + \frac{{ℏ}^{2} {π}^{2}}{2 {R}^{2}} ( \frac{1}{{m}_{e}} + \frac{1}{{m}_{h}} ) - \frac{1.8 {e}^{2}}{e R} \qquad ( 3 )$$

where mₑ and mₕ are the effective masses of electron and hole of quantum dots and ε is the bulk optical dielectric coefficient.

$${E}_{G}^{+} ( eV )$$

is the band gap energy,

$${E}_{g}^{b u l k}$$

band energy of bulk materials.

The Brus's model has been widely used to predict the relationship of QDs sizes, wave frequency shift, optical property of metal oxides. As listed in Table 1 in case (a), a laser excitation will generate a maximum SWIR absorption at 1212 (nm), resulting in a quantum efficiency (QE) of 30.2(%). In the case of a distributed feedback, laser with a central wavelength of 1580 (nm) as the laser source, the signal to noise ratio (SNR) of the wavelet-filtered spectrum improved. As the radium of QDs increases, the SNR absorption tends to shift to the long wavelength having a maximum of CO₂ absorption at 2000 (nm). However, the QE will be low for the SWIR camera imaging as demonstrated in case (d) in the listed Table 1 in the case (d). Special SWIR cameras should be used for determining the image of the SWIR spectrum.

In the case (b), the quantum efficiency (QE) is about 20(%) at wavelength in a range from 1310 to 1940 (nm), the QD size seems to have less influence on QE (%). In contrast, in the case (c), the size of QD from 1^{st} day to 10^{th} day doubled. The absorption wavelength also varies somewhat.

Recently, sulfide QDs have attracted broad attention in the field of bio-based material's sensing. Since the SWIR absorption bands of water, fatty acids, proteins are overtones and combination bands of biomaterial's vibrations, bonds connecting atoms are dominated by signals from involving hydrogen atoms such as O—H, C—H, and N—H (Proteins). As a matter of fact, the radiative energy transfer in these materials from electronic excitation to vibration absorption can be predicted. SWIR emitters have been developed with organic dyes as emitting probe and IR gas sensors. For example, experimental tests show that the matrix of lignin anchored with carbon dots could be quantitatively predicted on their plasmonic resonant of the capacitors from the dopped carbon dots.

TABLE 1
Comparative Examples of Enhancing the Performance of SWIR Emitters

| | | Types of | QD Radium | Absorption | QE | |
|---|---|---|---|---|---|---|
| Order | Technical Aprpoach | Products | nm | nm | % | Reference |
| 1 | Ag 2 S (Cationic dopping | QDs | 2.7 to 2.8 | 1212 | 30.2 | (a) |
| | with Pb + 1) | | | | | |
| 2 | Ag2 Se (Silver Selenide) | | Bohr Radium 2.9 | | | (b) |
| | T1 | QDs | 2.95 | 1310 | 20 | |
| | T5 | | 6.01 | 1940 | | |
| 3 | Ag Colloid particles (1st day) | QDs | 4.1 | 410 | 16.78 | (c) |
| 4 | (10th day) | QDs | 8.1 | 414 | | |

| | | | | |
|---|---|---|---|---|
| 5 | Monitoring CO2 absoprtion | 1570/2000 | [symbol] | (d) |
| | with two peaks | | | |

Note:
(a) Lin, H. H., Z. O. Tian, D. L. Zhang, D. W. Pang, 2018, Small/Volume 14 [symbol] issue 11, 1703290.
(b) Motnas, H., ET AL., 2023, Colloids and Surface A: Physicochemical and Engineering aspects, Volume 674, Oct. 5.
(c) Baset, S., H., Akbart, H., Zeynalt, M. Shafie, 2011, Digest Journal of Nanomaterials and Biostructures, Vol 6, no. 2, 709-716.
(d) Chong, X., E. LI, and K. Sauire, and A. X. Wang, 2016, Applied Physics Letters, 108, 221106. [symbol]
indicates data missing or illegible when filed

SWIR emitters have been used to monitor greenhouse gases such as CO₂ and methane flume in atmosphere and geological survey for identifying mineral deposits, however, surprisingly, none of the disclosed public reports show that SWIR can be used for monitoring CO₂ emissions of CCS in sequestration due to lacking an understanding on how the SWIR works with imaging.

The performance of the QDs products is not only dependent upon individual dots but also the assembly of these dots. The conductivity of dopped carbon dots might be described as electrons hopping from one excited QDs to the nearest neighbor. As two or more QDs are packed in a close pattern to each other, their wave functions (r) can be expressed in terms of coupling energy as described in equation 4:

$$π Γ = exp ( - 2 πΔ x \sqrt{\frac{8 m * Δ E}{{ℏ}^{2}}} ) \qquad ( 4 )$$

Where m* is the electron effective mass, ΔE, and Δx is the height of the tunneling barrier and shortest edge-to-edge distance between the nanoparticles, respectively.

Besides the quantization and electron hopping effect, coupling energy is also related with dipolar contribution from the piezoelectric effect of the substrate materials such as quartz. The coupling of mechanical stress and strain of the Piezoelectric substrates can be correlated with their dielectric and magnetic effect as prescribed with equations 5 and 6 in general,

$$( \begin{matrix}ε = {S}_{H} σ + {d}^{T} H & … & … & ( 5 ) \\ B = d ϵ + {μ}_{0} {μ}_{r} H & … & … & ( 6 )\end{matrix} )$$

where ϵ is the strain tensor, σ is the stress tensor, B is magnetic flux density, H is magnetic field, SH is material compliance matrix, d (dimension 3×6 is the piezomagnetic coupling matrix, μr is relative permeability of material, and μ₀ is the permeability of vacuum.)

The sensitivity for CO₂ monitoring in open air has been attributed to the electro-magneto resistive coupling. Controlled-source electromagnetic for CO₂ storage monitoring has been considered as successful, however, an application of the above QDs technology to enhance the monitoring function of CO₂ emission for CCS operation has not been reported. For a CCS VI well before getting a permit, a better monitoring system has been sought with the following desirable features: 1) hardening permanent sensor arrays to last for 5 to 30 years; 2) design permanently installed arrays; 3) design of field acquisition to monitoring changes at the boundaries of the CO₂; 4) use of capacitively coupled electric field elements, 5) use of casing electrodes where the well casing in an observation or monitoring well in the injection zone used as an electrode; 6) pre-digitalized and averaged data collection.

In response to the above requirements, a conceptual proof of design and fabrication for a product of quantum dots sensing contrast agent (QDSCA) comprised of hybrid quantum dots, coupled with an integrated digital transducer (IDT) of a thin layer semiconductor wired to a micro RF antenna, deposited on the surface of a piezoelectric substrate, and encapsulated with a hydrogel polymer coating in a multi-sensing mode. The following issues have been given great consideration in the designs:

Sensitivity: Monitoring CO₂ plume is essential for mitigating the risk of CO₂ leakage with technical approach of EM and seismic survey, however, both methods affected by the wave frequency (f) since the penetration depth (d) for an electromagnetic (EM) is a function of EM wave frequency expressed as equation 7:

$$d = 503 \sqrt{\frac{E}{f}} \qquad ( 7 )$$

where E is the rock stiffness, and f is the EM frequency.

It is obvious from equation 7 that if a high frequency (f) of wave function creates better imaging, it will reduce the penetration depth (d) greatly. A match of frequency (f) with the 1^{st} natural frequency of QDSCA is required to have a delineated imaging picture. The first natural frequency of QDSCA can be expressed as equation 8:

$${f}_{natural} = \frac{1}{2 π} \frac{{λ}^{2}}{{( \frac{D}{2} )}^{2}} \sqrt{\frac{{T}^{2}}{12 ( 1 - {v}^{2} ) ρ}} \qquad ( 8 )$$

where T is the height of the disk, v is the Poisson ratio, ρ is the density of QDSCA, D the diameter of the disk, λ² constant of the 1^{st} and 2^{nd} order of vibration frequencies, λ²(1^{st})=5.62, λ²(2^{nd})=12.4.

The wavelength (v) can be calculated with equation (9):

$$v = \frac{c}{f} \qquad ( 9 )$$

where c is the speed of wave function, v is the wavelength of electromagnetic field.

A comparative study of the penetration depth (d) and saturated CO₂ sensitivity based upon the equation from 7 to 9 demonstrates that CO₂ molecules in gas state for SWIR signals have a special QDs absorption at a wavelength of 2000 (nm) while in saturated brine state of d=5.64 (m), in rock solid 2976 (m). The corresponding signal frequency in gas states 150 (MHz), in saturated brine 40 (MHz), in rock solid 30 (MHz). The sensitivity (f) of CO₂ SWIR signal in gas state is the highest, in brine solution intermediate, in rock solid the lowest. In RF/SAW in gas, liquid, and state excellent. Clearly, the RF/SAW is the best way to transmit the CO₂ emission with an estimated penetration distance of 15 (km) 20 (kHz) in air and brine of 0.08 (m), and 2976 (m) in solid rock. Signals generated by QDSCA in terms of penetration depth (d) is 5 km in the case of SWIR gas state, 6 km in brine solution, and 2.6 km in rock solid. For photoacoustic and RF/SAW gas state, 6 km in brine solution, and 2.6 km in rock solid. For photoacoustic and RF/SAW wave energy, the preferred transfer media is only limited to rock solid.

Real-Time of Onsite 24/7: In tools used for geological surveys, localized rocks vibrated under small earth quickly or the drilling bore-head movements in the downhole direction are required. The signal to noise ratio (SNR) is low due to the low amplitude of vibration frequency and high noise signal by the limitation of moving instruments. Data collected from geological surveys only represents the typical geological events occurring at spatial time, the collected data is discrete. The data collected from QDSCA are continuous and consist of imaging frames generated by controlling the timing of the laser illumination, which allows for uninterrupted monitoring of CO₂ plume profiles. Carbon dioxide will be injected throughout the drilling process. After CO₂ injection, progression of the plume front and CO₂ concentrations can be monitored and measured in real time.

Multi-sensing Mode: Current EM methods have limited resolution due to single sensing. The QDSCA product converts high-frequency signals to low frequency while maintaining strong EM signal penetration, integrating SWIR, PAW, and RF/SAW sensing into one platform.

Unmatched Performance: Since QDSCA illuminates within the IR range from 1000 (nm) to 2500 (nm), it can penetrate fog, smoke, and dust. It will be a perfect option in dealing with CCS in downhole conditions in comparison with the regular IR-LEDs option.

Raw Materials for CCS Well Operation: The substrate used for fabricating the device is high purity silica sand or manufactured silicon, widely available in U.S. The AquLuMax™ solution is environmentally friendly multifunctional additives that can be applied by simply mixing it with CO₂. The report on the greenhouse program for monitoring, verification, and accounting (MVA) will be considered as part of an environmentally sustainable approach.

Tunability: The wavelength of quantum dots can be modified by altering their frequency and intensity.

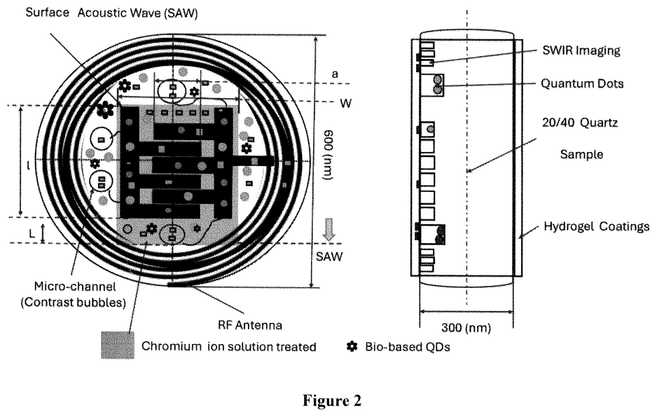

It is the purpose of this invention to measure the CO₂ concentration in situ in the carbon and storage (CCS) operation as an emitter as shown in FIG. 2, composed of (a) a microcrystalline silica, b) a metallic layer, c) tunable channels filled with a metal oxide of QDs (2-c1) and microlenses (2-d2) made of quantum dot's arrays, (d) conductive wires inserted with carbon QDs (2-b2) encapsulated in a hydrogel polymer coating as a signal emitter with unique identities assigned to each particle.

An integration of

$${V}_{p}^{m}$$

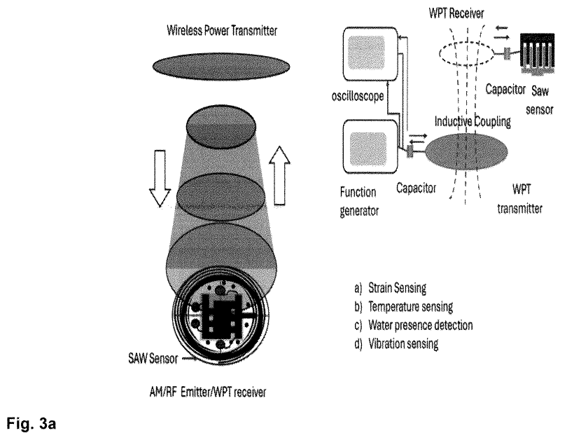

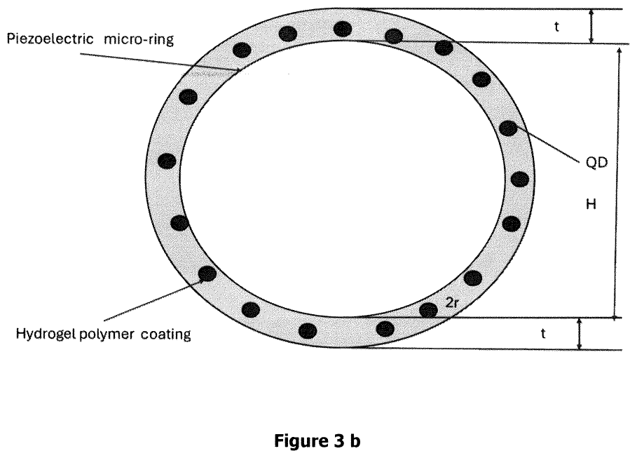

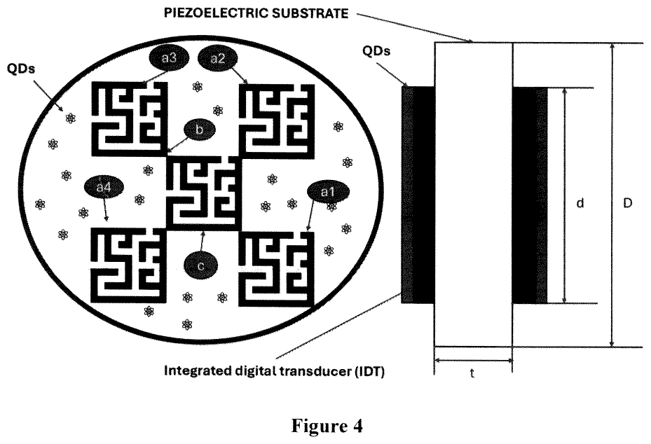

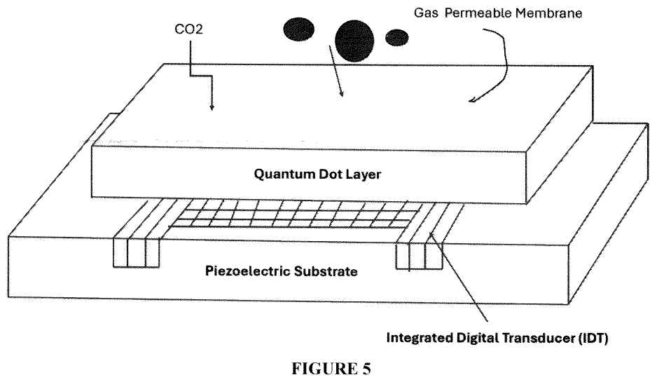

into CSEM model will create an onsite 3D CO₂ emission model in 24/7 mode. The drift of CO₂ plume profile from geological surveys' data can also be determined. The design of a QDSCA product based upon the above strategy shown in FIG. 3, comprised of a surface acoustic wave (SAW) chip with an interdigital transducer (IDT) deposited on a temperature and vibration. Bio-based carbon dots will be randomly incorporated in the production of QDs. Since quartz can convert mechanical energy to electrical energy, QDSCA can vibrate itself.

Combining quantum dots, RF systems, and SAW transducers offers an innovative method for monitoring environmental CO2 emissions. This hybrid platform leverages the unique optical properties of QDs, the dynamic modulation capabilities of SAWs, and the control precision of RF electronics to enable compact, high-sensitivity CO₂ sensors.

As previously stated, quantum dots offer size-tunable photoluminescence and high surface-to-volume ratios, making them ideal for detecting CO₂ molecules trace. Quantum dots functionalized with amine or carbonate ligands, or encapsulated proteins, demonstrate measurable shifts in emission upon binding with CO2. Their nanoscale footprint supports integration into micro- and nano electronic systems.

SAWs produced on piezoelectric substrates like quartz and LiNbO3 create localized strain and electric fields that affect QD emission. This acousto-optic interaction improves sensitivity and facilitates real-time signal transduction. Studies have demonstrated dynamic control of QD-like emitters using SAWs in nanowire heterostructures and 2D materials, validating the feasibility of such coupling.

RF circuitry drives SAW generation and enables frequency tuning for optical QD interaction. It also facilitates signal readout and feedback control, forming a closed loop sensing system. This integration supports miniaturization and remote operation, critical for distributed CO₂ monitoring networks.

Conceptually, an emitter of monitoring system for CO₂ emission in a 24/7 onsite model, is composed of multilayered structure as 1) SAW device on a piezoelectric substrate (like LiNbO₃ or Quartz); 2) electrode configuration for generating and tunning acoustic waves 3) possibly a microfluidic or CO₂-permable interface.

All of these layers are integrated into a uniform entity with quantum dot thin films deposited on top or embedded in an intermediate optical waveguide or suspended membrane aligned with the SAW path surface functionalization of QDs to selectively bind CO₂ leading to photoluminescence changes; coupled with SAW-driven modulation of QD fluorescence via strain-optic or piezoelectric field effects. RF feedback loop will allow the SAW frequency coupled with QD resonances, optical fiber/wavelength guide in-plane for excitation and emission collection. FIG. 3 shows a typical emitter, consisting of a quantum dots layer, gas-permeable membrane, digital transducers, and RF circulatory boards.

The quantum dots, consisting of a metal oxide frame (MOF), located on top of piezoelectric layers made from semiconductor materials such as Al0.3Ga0.7As, Ag, Cu, CuO, Ag₂S, can be used to obtain the SWIR spectrum based upon the measured data from the SWIR spectrum. Detailed examples illustrating the performance of QDSCA are presented below.

### EXAMPLES

Example 1: A pre-cut quartz substrate was treated with chromic ion solution, then screen printed with 15-20 micron thick, 25-micron wide silver or copper paste to prepare the products. As defined in FIG. 2, the samples have an IDT aperture a=220 (micron); SAW propagation length L=250 (micron); I=100 (micron) and w=300 (micron). A radio frequency antenna was screen printed on the drum surface. A 30-micron wire forms an Archimedean spiral between the fifth and eighth circles, functioning as an RF antenna. Screen printing can utilize a variety of synthetic methods for production. Several synthetic methods are available for the chemical synthesis of silver and copper-based quantum dots. To prepare CuO QDs, mix 0.06 M CuCl2·2H2O solution with ammonia in a 9:1 ratio to keep the pH around 9, dipping the substrate in the solution for 20 seconds, then heated to 85 °C. Distilled water was used to wash the treatment at room temperature for 20 seconds to remove loosely attached copper species. This process was repeated forty times under ultrasonication. The samples were annealed at 500 °C for 4 hours in air.

Fabrication: A 20/40 silica sand will grinded into a drum size in diameter of 600 (micron) and thickness of 300 (micron) as previously described in IDT section. A procedure for preparing QDs of CuS and/or Ag2S will include: 10-nm sized CuxSy nanoparticles were created by mixing sodium citrate with an aqueous solution of CuCL₂ and Na₂S as precursor. Alternatively, L-ascorbic acid was mixed with CuCL₂ precursor within a diameter of 3.5 to 4.0 (nm). A procedure for preparing QDs with a reversed emulsion includes: The substrate was coated with AquLuMax™, then, 10 (gram) of coated materials were immersed in 50 ml of acetone solution (0.05 M), decanting the solution, and drying the samples before assessing the products. The quantum dots sizes and distribution could be controlled by manipulating the solution concentration and reaction time.

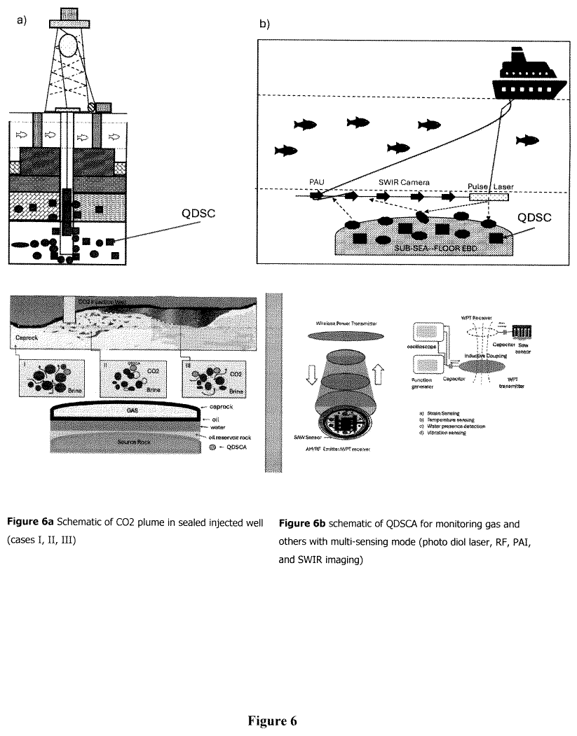

Application: Each QDSCA disk can be considered as one unique emitter with unique ID identified by the SWIR imaging handled in case by case shown in FIG. 6a: In case I, all QDSCA immersed in the brine phase and directly interacted with the dissolved CO₂, in II, SOME OF QDSCA particles immersed in brine, some at the interface between brine and CO₂ plume, in III, the adsorbed plume gas surrounding the QDSCA. As shown in FIG. 6b, individual QDSCA emitters can be stimulated by a photodiode laser having maximum SWIR frequencies between 1000 and 2500 (nm). Frequency shift from QD size control (equation 1) and quantum tunnelling (equation 2) might be initialized due to the coupling of QDs distribution. It is believed that the disclosed concept present here is unique. The developed products should allow us to have a robust system for monitoring CO₂ plume in the CCS operation.

Like resin coated proppants as flow back control agents, coated QDSCA could be injected at a dose level of 1.0 to 2.0% over CO₂ wt. (%) in EOR-CO₂ fracking or pumped into the well at a dose level of 6.0 to 20.0% over proppants by weight percentage in the VI well and unconventional hydraulic fracking operation.

The Brus's model has been widely used to predict the relationship of QDs sizes, wave frequency shift, optical property of metal oxides. Sulfide (QDs) have attracted broad attention in the field of bio-based material's sensing. Since the SWIR absorption bands of water, fatty acids, proteins are overtones and combination bands of biomaterial vibrations, bonds connecting atoms are dominated by signals from involving hydrogen atoms such as O—H (water, carbohydrates), C—H (lipids), and N—H (proteins).

SWIR emitters were developed with organic dyes as emitting probe and IR gas sensors. For example, experimental tests show that the matrix of lignin anchored with carbon dots could be quantitatively predicted on their plasmonic resonant of the capacitors from the dopped carbon dots, useful to monitor greenhouse gases such as CO₂ and methane flume in atmosphere and geological survey for identifying mineral deposits. Similarly, monitoring CO₂ emission in CCS in sequestrations due to lacking in understanding of how the SWIR works with imaging is possible in the field.

It is obvious from equation 7 that if a high frequency (f) of wave function creates better imaging, it will reduce the penetration depth (d) greatly. A match of frequency (f) with the 1^{st} natural frequency of QDSCA is required to have a delineated imaging picture.

Example 2 A hydrogel coated micro-ring sample as shown in FIG. 3 prepared with parameter defined in Table 2.

TABLE 2
Parameters Used in Numerical Simulations

| Parameters | Values | | Units | Note |
|---|---|---|---|---|
| D | 40.0 | | Micron | Measured |
| d | 6.1 | | Micron | Measured |
| t | 0.2 | | Micron | Measured |
| | Micro-ring quartz | Hydrogel coating | | |
| n (RI) | 1.45 | 1.41 | m − 1 | |
| Absorb. | 0.0156 | 2.3 | m − 1 | |
| C | 740 | 1300 | J/KG/K | |
| P | 2650 | 970 | KG/M³ | |
| K | 1.38 | 0.21 | W/m/K | Constant |
| dnⱼ/dT | 1.2 × 10⁻⁵ | −1.0 × 10⁻⁴ | k⁻¹ | Constant |

The prepared coated micro-ring sample had a diameter of 42.0 (micron), inner diameter of hole of 6.0 (micron), the hydrogel coating 0.20 (micron) in thickness. In a typical laboratory experimental setting, a tunable external cavity diode laser in 1450 nm band was utilized to implement a fine scan of the laser wavelength around the prepared sample. The transmitting light was detected by a low noise detector subsequently connected to an oscilloscope to monitor the transmission spectra of the prepared micro-ring quartz samples.

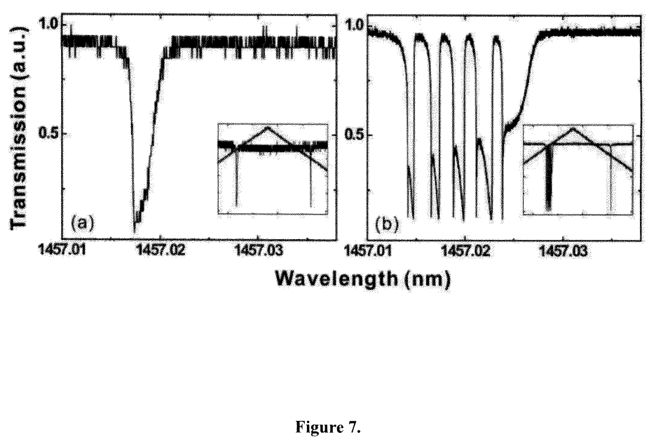

The measured intrinsic quality factor of the fundamental sample's 1^{st} resonator is about 3.8×10⁶, measured at a low input power of 0.7 mW to avoid thermal effects. In contrast, at a high input power of 3.1 mW, the transmission spectrum became broadening, and a fine structure characterized by oscillations within the linewidth of the resonance. FIG. 7 shows the characteristic behavior of the SWIR spectrum of coated samples for monitoring the sample's thermal environmental behavior.

Clearly, the spectrum fine structure of linewidth broad is controlled by the input power that can be manipulated manually. As such, the shift of frequency of the SWIR spectrum can be used to monitor the CO₂ control concentration at the wavelength between 900 to 2500 (nm). Also, the shifting of the absorption peak around 2000 (nm) provides the second peak for characterizing the CO₂ concentration in the second modulus.

Based upon the disclosure present here, it is therefore demonstrated that the objectives of the present inventions are accomplished by the chemical components and structural combination of the designated coated piezoelectric disk and micro spherical configuration with special procedures and compositions of matters., and preparation of methods, its applications and identified benefits for the refracting of hydraulic fracking, CO₂-EOR fracking, and CO₂ monitoring in type VI well applications. It showed to be understood that the selection of the special piezoelectric dielectric materials, hydrogel polymers, nano particles, and other components made up by wt. % can be determined by one having regular ordinary skill in the art without departing from the spirit of the invention herein disclosed and described. It should therefore be appreciated that the present invention is not limited to the specific embodiments described above, but includes variation, modification, and equivalent embodiments defined by the following claims.

This invention claims:

1. Quantum dots sensing contrast agents comprised of basic elements:
1) quantum dots in a diameter from 1.0 to 390 (nm),
2) micro/macro channels from 5 to 100 (micron),
3) conductive wires with an integrated digital transducer (IDT) logic,
4) fabricated on a piezoelectric substrate in which, the quantum dots arrays excited by a laser irradiating short wave Infrared (SWIR) between 900 to 1700 (nm) scanned by an imaging system, the micro/macro channels filled with quantum dots effectively absorbing CO₂ gas, resulting in a contraction and expansion of the locked gases in the channels that convert the SWIR wave energy into the ultrasonic energy to expand/compress the encapsulated hydrogel polymers as contrasting bubbles visualized by an ultrasonic device, transmitting the needed radio frequency (RF) signal via the piezoelectric substrate in the subterrain and wet seabed environment for monitoring CO₂ plume concentration in multi-sensing mode onsite continuously.

2. The basic elements of claim 1, wherein quantum dots arrays, made of semiconductive materials, aligned together in an array matrix of x*y (x=1, . . . n, y=1,2 . . . , m, n, integrated numbers), in a size of x and y from 1 to 200 (nm) on each dot, coated on the piezoelectric substrate in a number of n and m.

3. The basic elements of the piezoelectric substrate of claim 2, wherein the fabricated micro channels and/or microlens arrays drilled within the piezoelectric substrate within a dimension in the diameter between 1 to 200 (micron) and in depth of less than 250 (micron).

4. The basic elements of claim 3, wherein conductive wires are intertwined and spiraled into coils within the quantum dot's arrays implanted on the piezoelectric substrate in a size of 10 to 2000 (micron) in length and 5 to 100 (nm) in diameter.

5. The basic elements of claim 4, wherein the fabricated micro channels microlens arrays made of etching the metallic thin layered coating deposited on the portion of the substrate within a thickness of less than 500 (nm).

6. The basic elements of claim 5, wherein the electric conductive wires designated in such a way that the up conversion of a laser excited infrared wavelength range of quantum dots between 800 to 899 (nm) easily move to short wave infrared spectrum at 900 to 2500 (nm) detectable by a short-wave infrared imaging (SWIR) system, irradiated at 1200 (nm) and 1650 (nm) with quantum dots arrays, at least one of quantum dots made of silver sulfide (Ag₂S) or a modification of silver sulfide compounds, or quantum dot's domains sized within less than 50 (nm).

7. The basic elements of claim 6 wherein short-wave infrared (SWIR) waves with peak absorption within 1950-2150 nm, having maximum CO₂ micro-channel chamber expansion and contraction by converting maximum absorption of infrared thermal energy into ultrasonic wave energy.

8. The basic elements of claim 7, wherein the micro-channels partially filled with quantum dots sized less than 200 (nm) to achieve the maximum thermal energy conversion, resulting in the maximum magnitude of ultrasonic wave sensing signals as a contrast agent detected by a non-destructive ultrasonic device.

9. The basic elements of claim 8, wherein the concentration of CO₂ in the tested subterrain region can be determined on the plasmonic absorption of CO₂ gas within the top surface of the substrate filled with quantum dots and CO₂ bubbles.

10. The basic elements of claim 9, wherein the substrate is a proppant including the particles: a) microcrystalline piezoelectric silica particle and/or dust, b) bauxite, and c) ceramics proppants in size of diameters less than 40/70 mesh.

11. The basic elements of claim 10, wherein the piezoelectric substrate is partially coated with a thin metallic layer with pure copper (Cu), or other metals (Au, Aluminum, Ag, Pt), or metallic inorganic compound of graphene and graphite, or/and organic conductive polymers, having excellent conductivity to the electron with a thickness of metal layer less than 1000 (nm).

12. The basic elements of claim 11, wherein the metallic layer fabricated with special sized microchannels having shaped in a slit with a dimension of each slit in length of 50-100 (nm), in the width of 40-60 (nm), in depth of 1000-1500 (nm).

13. The basic elements of claim 12, wherein the fabricated slits aligned together in pairs to form slit matrix (4×4, 8×8, 12×12) to create micro-channels that can hold the CO₂ gas within with an integrated logic networked by metallic wires coated with quantum dots with special chemical components on the surface of substrates and within the microchannels.

14. The chemical components of claim 13, wherein the microchannels partially filled with quantum dots made of materials of copper sulfide (Cu₂S), silver sulfide (Ag₂S), gold sulfide (Au), organic sulfide materials having a structure R′S=SH or RS=SR (R=alkyl functional groups of organic polymeric hydrocarbon group).

15. The chemical components of claim 14, wherein it can be dopped with a partial modification to the inorganic or organic compound materials with the following inorganic chemical containing the cations: Ca⁺², Fe⁺³, Ba⁺², Si⁺⁴, Ti⁺⁴, Ta⁺¹, Zn⁺², B⁺¹, Mo⁺², Mn⁺⁴, Er³⁺, Nd³⁺, Yt³⁺, Li⁺, to tailor the adjusted SWIR peaks for laser exciting and irradiating the special SWIR wave signals.

16. The basic chemical components of claim 15, wherein it can further be prepared by an in-situ formation of chemical reaction with cationic or ionic implantation by blending with hydrogel polymer of protein or amino-acid components to generate quantum dots randomly or aligned in an array.

17. The chemical components of claim 16, wherein the topological surface textured nano-patterns resulting in special irradiation of SWIR energy bands within from 1000 to 1700 (nm) are detectable with a SWIR imaging an analytical system, further transferring the SWIR into a radio frequency (RF) wave transmitted to a desirable receiver with a fingerprinting scan identification code.

18. The chemical components of claim 17, wherein the designated micro-device can emit the spectrum in multiple modes including radio frequency, short-wave infrared spectrum, and surface acoustic wave (PAW) spectrum.

19. The chemical components of claim 18, wherein the quantum dots interconnected with a 3D printing wire system prepared with copper or silver pastes printed on the proppant's particle surface, the 3d printing electric wire patterns are in a double-spiraled and mended pattern that enhances the magnitude and signal strength of SWIR irradiation quantum efficiency (QE) by over 50%, the generated radio frequency from the wire network on the printed logic emitted into the environment in a range between from 10 to 100,000 (Hz).

20. The chemical components of claim 19, wherein the data originated from both SWIR irradiation and ultrasonic wave energy can be transmitted and CO₂ deposition under sea water rock/shell to a central computation center for monitoring the CO₂ plume emission with the installed QDSC in situ in the scenario of carbon capture sequestration (CCS) operation within a distance between 1 and 3000 meter sealed under a water resistance packaging environment.
